(19) United States
(12) Patent Application Publication
(10) Pub. No.: US 2016/0184131 A1
(43) Pub. Date: Jun. 30, 2016

(54) HEATING AND COOLING THUMB WRAP

(71) Applicants: Chet Todd Miller, Plant City, FL (US); Sally Ann Miller, Plant City, FL (US)

(72) Inventors: Chet Todd Miller, Plant City, FL (US); Sally Ann Miller, Plant City, FL (US)

(21) Appl. No.: 14/585,302

(22) Filed: Dec. 30, 2014

(51) Int. Cl. A61F 7/00; A61F 5/01

(52) U.S. Cl. CPC A61F 7/00; A61F 5/013; A61F 2007/0038; A61F 2007/0036; A61F 2007/0035

(57) ABSTRACT

A therapeutic thumb wrap is provided. The thumb wrap includes a body having an inner fabric layer and an outer fabric layer attached together by a seam. At least one pocket is formed in between the inner and outer fabric layers. A plurality of dried seeds is disposed within the pocket. The body includes a thumb portion with a first side extending downward from the thumb portion and a second side opposite the first side extending downward from the thumb portion. The first side and the second side are sized to wrap around a wrist of the user. A connector may releasably attach the first side to the second side, securing the thumb wrap to the user's wrist.

### BACKGROUND OF THE INVENTION

The present invention relates to a thumb wrap and, more particularly, to a thumb wrap for heating or cooling the thumb or wrist.

The median nerve is a nerve in humans and other animals in the upper limb. It is one of the five main nerves originating from the brachial plexus. Injury of median nerve at different levels causes different syndromes with varying motor and sensory deficits. Carpal tunnel syndrome (CTS) is a median entrapment neuropathy that causes parethesia, pain, numbness and other symptoms in the distribution of the median nerve due to its compression at the wrist in the carpal tunnel. It appears to be caused by a combination of genetic and environmental factors. Some of the predisposing factors include: diabetes, obesity, pregnancy, hypothyroidism, and heavy manual work or work with vibrating tools. Applying heat or cold may help relieve the symptoms of CTS.

As can be seen, there is a need for a therapeutic thumb wrap apply a hot or cold temperature to the wrist.

### SUMMARY OF THE INVENTION

In one aspect of the present invention, a thumb wrap comprises: a body comprising an inner fabric layer attached to an outer fabric layer forming at least one pocket in between, and a plurality of seeds disposed within the pocket, wherein the body comprises a thumb portion sized to fit around a thumb, and a first side extending downward from the thumb portion, and a second side opposite the first side extending downward from the thumb portion; and a connector releasably attaching the first side to the second side.

In another aspect of the present invention, a method of treating a hand or wrist injury comprises: providing a thumb wrap comprising a body comprising an internal pocket, and a plurality of seeds disposed within the internal pocket, wherein the body comprises a thumb portion, a first side extending downward from the thumb portion, and a second side opposite the first side extending downward from the thumb portion; heating or cooling the thumb wrap; placing the thumb portion of the body over a user's thumb; wrapping the first side and the second side around the user's wrist; and releasably attaching the first side to the second side, thereby securing the thumb wrap to the user.

These and other features, aspects and advantages of the present invention will become better understood with reference to the following drawings, description and claims.

### BRIEF DESCRIPTION OF THE DRAWINGS

FIG. 1 is a bottom view of an embodiment of the present invention shown in use;

FIG. 2 is a top view of an embodiment of the present invention shown in use;

FIG. 3 is a bottom view of an embodiment of the present invention;

FIG. 4 is a top view of an embodiment of the present invention; and

FIG. 5 is a section view of an embodiment of the present invention along line 5-5 in FIG. 3.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a reusable thumb wrap to deliver heat or cold to a thumb or wrist. The present invention may be shaped to apply hot or cold to the area affected while allowing a degree of use of the hand. The wrap may cover the thumb and extend downwards, exposing a large portion of the palm and the back of the hand. The fabric is cut to the pattern, sewed to the fleece material and filled with a dried seed. Once heated or cooled, the dried seed may retain the temperature for an extended therapy.

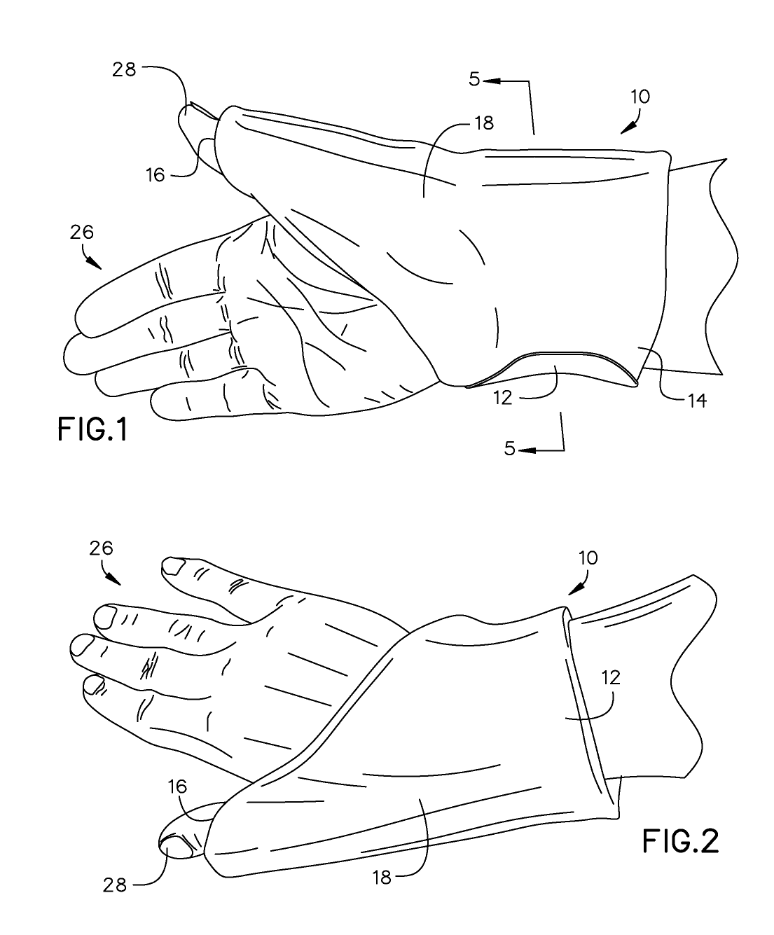

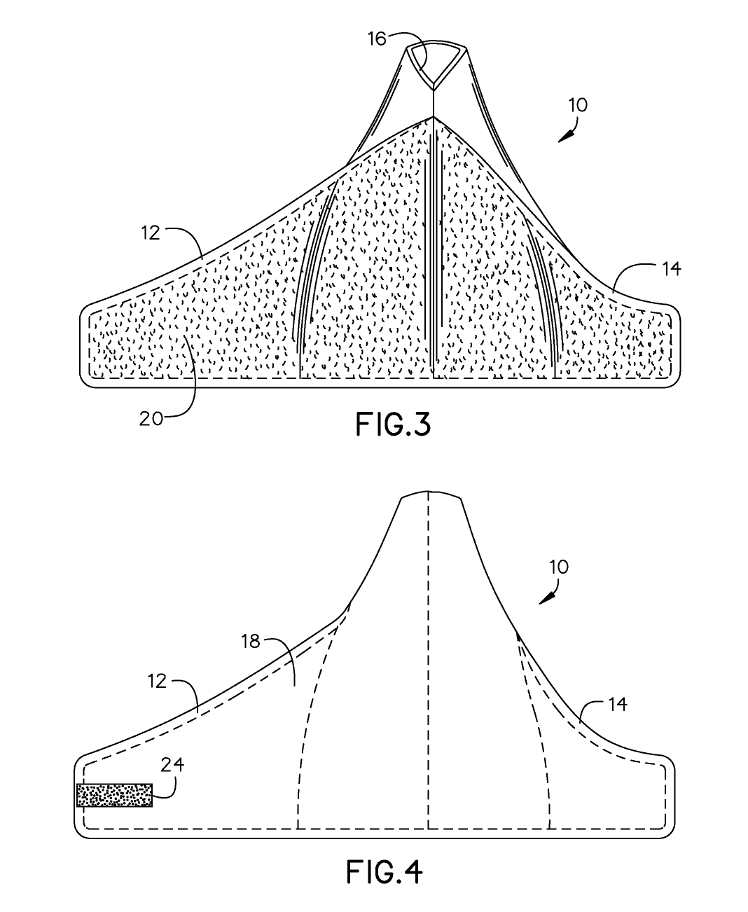

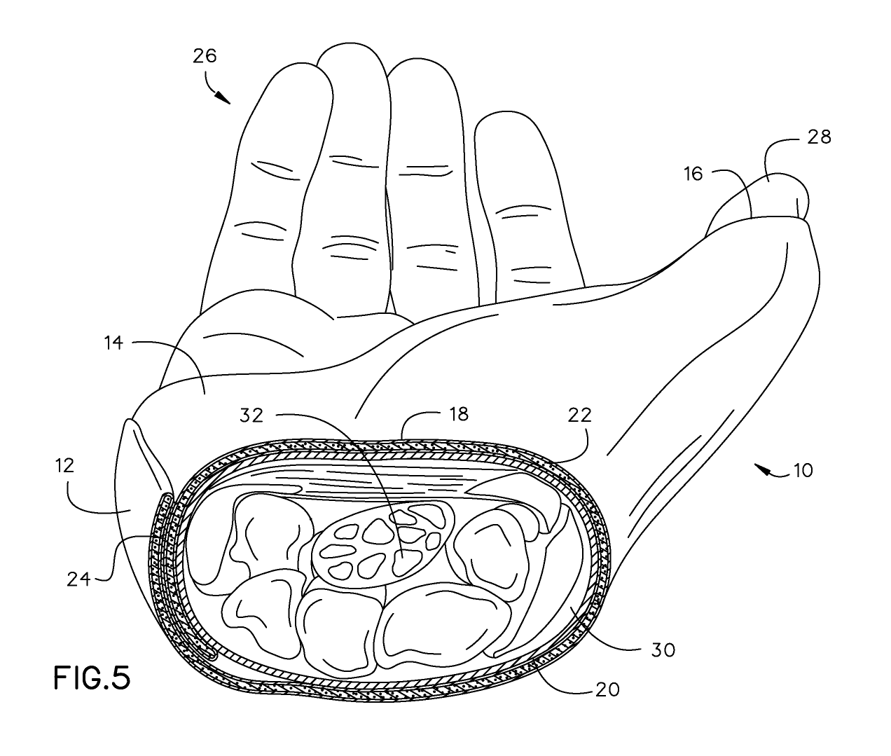

Referring to FIGS. 1 through 5, the present invention includes a thumb wrap 10. The thumb wrap 10 includes a body having an inner fabric layer 20 and an outer fabric layer 18 attached together by a seam. At least one pocket is formed in between the inner and outer fabric layers 20, 18. A plurality of dried seeds 22 is disposed within the pocket. The body includes a thumb portion 16 with a first side 12 extending downward from the thumb portion 16 and a second side 14 opposite the first side 12 extending downward from the thumb portion 16. The first side 12 and the second side 14 are sized to wrap around a wrist 30 of the user 26. A connector 24 may releasably attach the first side to the second side, securing the thumb wrap 10 to the user's wrist 30. The present invention may provide relief to the median nerve 32.

As illustrated in the Figures, the thumb wrap 10 includes a top portion and a bottom portion. The first side 12 and second side 14 may taper downwards from the top portion to the bottom portion forming a narrow section where the first and second sides 12, 14 end. The ends of the first and second sides 12, 14 may releasably attach to one another by a hook and loop fastener, or other connector 24, such as but not limited to, a clip, a snap button, a pin, a tie, and the like. In certain embodiments the first side 12 may be attached to the second side 14 near the top portion. A thumb hole sized to receive a thumb 28 is formed through an upper rim of the attached first side 12 and second side 14.

The present invention may further include a plurality of seams connecting the inner fabric layer 20 and the outer fabric layer 18. The plurality of seams may be formed by stitching the layers 20, 18 together from the top portion to the bottom portion. The plurality of seams forms a plurality of pockets in between. The dried seeds 22 may be disposed within each of the plurality of pockets.

The thumb wrap 10 of the present invention may be constructed of various materials. For example, the inner fabric layer 20 may be a comfortable and warm material, such as a fleece material. The outer fabric layer 18 may be made of a cotton and/or polyester material. The dried seeds 22 may be a variety of seeds, such as grains and beans. For example, the dried seeds 22 may include flax seed and/or rice.

The present invention may further include a method of applying heat of cold to a thumb or wrist. Applying heat or cold may relieve pain from strain, such as carpel tunnel syndrome. The present invention may be used as heat therapy and cooling therapy. For example, the thumb wrap may be placed in the freezer for a period of time. The thumb wrap may be removed from the freezer, and the thumb portion may placed over the user's thumb. The first side and the second side may be wrapped around the user's wrist. The first side and the second side may then be releasably attached to one another, thereby securing the thumb wrap to the user. Alternatively, the thumb wrap may be placed in a microwave and heated up. The thumb wrap may be removed from the microwave, and the thumb portion may placed over the user's thumb. The first side and the second side may be wrapped around the user's wrist. The first side and the second side may then be releasably attached to one another, thereby securing the thumb wrap to the user.

It should be understood, of course, that the foregoing relates to exemplary embodiments of the invention and that modifications may be made without departing from the spirit and scope of the invention as set forth in the following claims.

What is claimed is:

1. A thumb wrap comprising:
a body comprising an inner fabric layer attached to an outer fabric layer forming at least one pocket in between, and a plurality of seeds disposed within the pocket,
wherein the body comprises a thumb portion sized to fit around a thumb, and a first side extending downward from the thumb portion, and a second side opposite the first side extending downward from the thumb portion; and
a connector releasably attaching the first side to the second side.

2. The thumb wrap of claim 1, wherein the first side and the second side are sized to wrap around a wrist.

3. The thumb wrap of claim 1, wherein the first side and the second side are connected together at a top portion of the body forming a thumb hole in between.

4. The thumb wrap of claim 1, further comprising a plurality of seams connecting the inner fabric layer and the outer fabric layer and running from a top portion to a bottom portion of the body, forming a plurality of pockets in between, wherein the plurality of seeds are disposed within each of the plurality of pockets.

5. The thumb wrap of claim 1, wherein the connector is a hook and loop fastener.

6. The thumb wrap of claim 1, wherein the seed is at least one of flax seed and rice.

7. The thumb wrap of claim 1, wherein the inner fabric layer comprises a fleece material and the outer fabric layer comprises a cotton material.

8. A method of treating a hand or wrist injury comprising:
providing a thumb wrap comprising a body comprising an internal pocket, and a plurality of seeds disposed within the internal pocket, wherein the body comprises a thumb portion, a first side extending downward from the thumb portion, and a second side opposite the first side extending downward from the thumb portion;
heating or cooling the thumb wrap;
placing the thumb portion of the body over a user's thumb;
wrapping the first side and the second side around the user's wrist; and
releasably attaching the first side to the second side, thereby securing the thumb wrap to the user.

9. The method of claim 8, wherein heating the thumb wrap comprises heating the thumb wrap in a microwave.

10. The method of claim 8, wherein cooling the thumb wrap comprises placing the thumb wrap in a freezer.
